(19) United States
(12) Patent Application Publication
(10) Pub. No.: US 2026/0069111 A1
(43) Pub. Date: Mar. 12, 2026

(54) ENDOSCOPE PROCESSOR, METHOD, AND COMPUTER-READABLE MEDIUM FOR CONTROLLING POWER SUPPLY TO, AND DISCHARGE FROM, AN ENDOSCOPE

(71) Applicant: OLYMPUS MEDICAL SYSTEMS CORP., Tokyo (JP)

(72) Inventor: Masaki KONDO, Tokyo (JP)

(73) Assignee: OLYMPUS MEDICAL SYSTEMS CORP., Tokyo (JP)

(21) Appl. No.: 19/384,187

(22) Filed: Nov. 10, 2025

Related U.S. Application Data
(63) Continuation of application No. PCT/JP2023/019116, filed on May 23, 2023.

(51) Int. Cl. A61B 1/00

(52) U.S. Cl. CPC A61B 1/00027; A61B 1/00114

(57) ABSTRACT

An endoscope processor includes a power source circuit configured to supply power to an endoscope, an endoscope connector, a discharge resistor between the power source circuit and the endoscope connector, a power conduction circuit between the discharge resistor and the endoscope connector, and a processor. The processor is configured to, in response to detecting connection of the endoscope to the endoscope connector, control the power conduction circuit to establish conduction between the endoscope and the discharge resistor, in response to starting power supply to the endoscope by the power source circuit, control the power conduction circuit to interrupt the conduction, and in response to resuming power supply to the endoscope after a stop, control the power conduction circuit to reestablish the conduction. When the conduction is established, charge accumulated in the endoscope is discharged through the discharge resistor.

### CROSS REFERENCE TO RELATED APPLICATIONS

This application is a continuation of International Application No. PCT/JP2023/019116 filed on May 23, 2023, the entire contents of which are incorporated herein by reference.

### BACKGROUND

### TECHNICAL FIELD

The present disclosure relates to an endoscope processor, a method, and a computer-readable medium for controlling power supply to, and discharge from, an endoscope connected to the endoscope processor.

### Related Art

An endoscope apparatus includes an endoscope including an image pickup device, and an endoscope processor that performs video processing on a captured image obtained by the endoscope. The endoscope processor converts the captured image into a video signal, which may be output to a monitor or recorded.

Many types of endoscopes are connectable to the endoscope processor. Various types of image pickup devices may be employed in the endoscopes. The endoscope processor is designed to detect a type of the image pickup device mounted in the endoscope and perform optimal driving according to the type of the image pickup device.

The endoscope is connected to the endoscope processor in a detachable manner, either directly or through an adapter. The endoscope receives power from the endoscope processor and supplies a captured image to the endoscope processor. In order to normally start up the endoscope apparatus, the endoscope processor needs to supply power to the endoscope at the time of the startup according to a startup sequence defined for each endoscope.

In order to properly execute the startup sequence, electric charge in a power source circuit of the endoscope needs to be discharged at startup. Therefore, it is necessary to ensure a sufficient period for ending the endoscope (hereinafter referred to as “end processing period”) so that the power source circuit of the endoscope is completely discharged at ending of the endoscope.

### SUMMARY

According to aspects of the present disclosure, an endoscope processor is provided, which includes a power source circuit, an endoscope connector, a discharge resistor, a power conduction circuit, and a processor. The power source circuit is configured to supply power to an endoscope. The discharge resistor is disposed between the power source circuit and the endoscope connector. The power conduction circuit is disposed between the discharge resistor and the endoscope connector. The processor is configured to, in response to detecting connection of the endoscope to the endoscope connector, control the power conduction circuit to establish conduction between the endoscope and the discharge resistor. The processor is further configured to, in response to starting power supply to the endoscope by the power source circuit, control the power conduction circuit to interrupt the conduction. The processor is further configured to, in response to resuming power supply to the endoscope after a stop, control the power conduction circuit to reestablish the conduction. When the conduction is established, charge accumulated in the endoscope is discharged through the discharge resistor.

According to aspects of the present disclosure, further provided is a method implementable on a processor of an endoscope processor. The endoscope processor includes a power source circuit, an endoscope connector, a discharge resistor, and a power conduction circuit. The method includes controlling, in response to detecting connection of an endoscope to the endoscope connector, the power conduction circuit to establish conduction between the endoscope and a discharge resistor. The power conduction circuit is disposed between the discharge resistor and the endoscope connector. The discharge resistor is disposed between the power source circuit and the endoscope connector. The power source circuit is configured to supply power to the endoscope. The method further includes controlling, in response to starting power supply to the endoscope by the power source circuit, the power conduction circuit to interrupt the conduction. The method further includes controlling, in response to resuming power supply to the endoscope after a stop, the power conduction circuit to reestablish the conduction. When the conduction is established, charge accumulated in the endoscope is discharged through the discharge resistor.

According to aspects of the present disclosure, further provided is a non-transitory computer-readable medium storing computer-readable instructions that are executable by a processor of an endoscope processor. The endoscope processor includes a power source circuit, an endoscope connector, a discharge resistor, and a power conduction circuit. The computer-readable instructions are configured to, when executed by the processor, cause the endoscope processor to, in response to detecting connection of an endoscope to the endoscope connector, control the power conduction circuit to establish conduction between the endoscope and a discharge resistor. The power conduction circuit is disposed between the discharge resistor and the endoscope connector. The discharge resistor is disposed between the power source circuit and the endoscope connector. The power source circuit is configured to supply power to the endoscope. The computer-readable instructions are further configured to, when executed by the processor, cause the endoscope processor to, in response to starting power supply to the endoscope by the power source circuit, control the power conduction circuit to interrupt the conduction. The computer-readable instructions are further configured to, when executed by the processor, cause the endoscope processor to, in response to resuming power supply to the endoscope after a stop, control the power conduction circuit to reestablish the conduction. When the conduction is established, charge accumulated in the endoscope is discharged through the discharge resistor.

### BRIEF DESCRIPTION OF THE DRAWINGS

FIG. 1 is a configuration diagram showing an endoscope apparatus employing an endoscope discharge method according to a first embodiment of the present disclosure.

FIG. 2 is a timing chart showing an endoscope discharge method in a comparative example.

FIG. 3 is a timing chart showing the endoscope discharge method in the present embodiment.

FIG. 4 is a flowchart showing the endoscope discharge method in the present embodiment.

FIG. 5 is a configuration diagram showing an endoscope apparatus employing an endoscope discharge method according to a second embodiment of the present disclosure.

FIG. 6 is a flowchart for describing the second embodiment.

### DESCRIPTION OF THE EMBODIMENTS

Depending on the state when the endoscope is ended (end condition), for example, a case where a hot swap is performed, the endoscope power source circuit may be in a state in which electric charge remains. In this case, in order to properly execute the startup sequence, startup of the endoscope processor needs to be delayed until the electric charge in the endoscope power source circuit is discharged.

Thus, for proper execution of the startup sequence, relatively long time needs to be ensured, as the end processing period and/or the time required at startup (hereinafter referred to as “startup time”). If the end processing period becomes long, the waiting time until the endoscope is removed becomes long, which lengthens the time the user is occupied. If the startup time becomes long, it takes a relatively long time for an image to be output from the endoscope at the startup, resulting in a problem that the examination may not be begun immediately.

As measures for solving such a problem, methods may be considered such as constructing a dedicated circuit for discharging an endoscope power source using a switch such as an FET, and providing a discharge resistor in the endoscope, in order to speed up the discharge time and improve the startup time and the end processing period (see Japanese Patent Application Laid-Open Publication No. 2021-122455). However, in such measures, a dedicated circuit must be newly added to the endoscope.

Hereinafter, embodiments of the present disclosure will be described in detail with reference to the drawings.

### First Embodiment

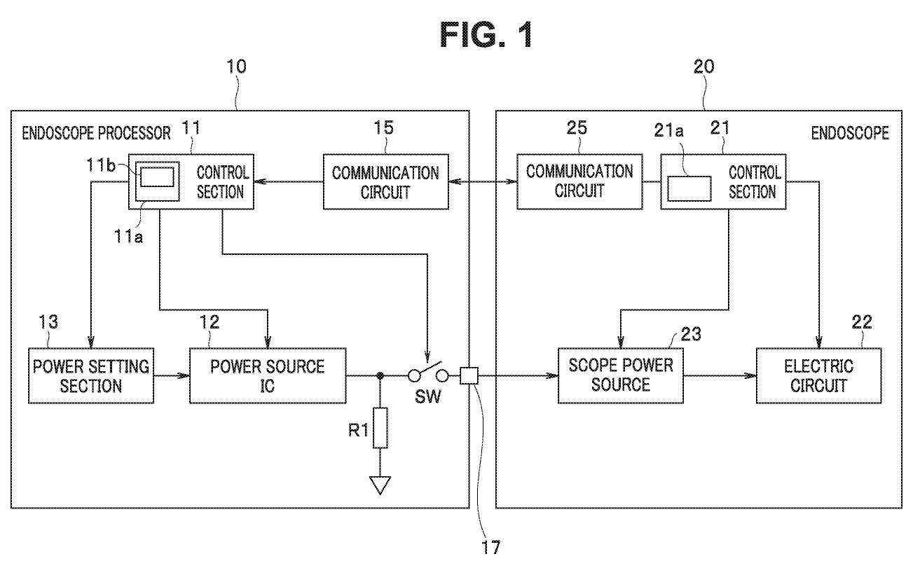

FIG. 1 is a configuration diagram showing an endoscope apparatus employing an endoscope discharge method according to the first embodiment of the present disclosure. The present embodiment is one that utilizes an existing discharge resistor provided in an endoscope processor to discharge a power source of an endoscope utilizing the idle time of the system operation, thereby enabling the startup time and the end processing period of the endoscope apparatus to be shortened.

In FIG. 1, the endoscope apparatus includes an endoscope processor 10 and an endoscope 20. A control section 21 is provided in the endoscope 20. The control section 21 may be configured with an FPGA (Field Programmable Gate Array), or may be configured with a processor using an unshown CPU or the like, to be capable of controlling each section according to a program stored in a memory.

The endoscope 20 is designed to be detachably attached to a connector of the endoscope processor 10. The endoscope processor 10 includes a detection section that detects whether the endoscope processor 10 is connected to the endoscope 20 (illustration omitted). Note that the present disclosure is not limited to this, and may have a structure in which an adapter is interposed between the endoscope processor 10 and the endoscope 20.

In the endoscope 20, an unshown image pickup device such as a CCD or CMOS sensor is placed at a distal end of an unshown insertion portion of the endoscope 20, and the endoscope 20 includes various electric circuits 22 including an image pickup device. Power to be supplied to the electric circuit 22 of the endoscope 20 is designed to be supplied from the endoscope processor 10.

A scope power source 23 of the endoscope 20 is controlled by the control section 21 to stabilize the power to be supplied to the electric circuit 22 in the endoscope 20. The scope power source 23 is supplied with power from the endoscope processor 10, and supplies a power source voltage to the electric circuit 22 based on the supplied power. The control section 21 is designed to operate by being supplied with the power source voltage from the scope power source 23 (illustration omitted), to drive and control the electric circuit 22. For example, the control section 21 performs control of a state of operations, such as exposure and readout, and causes the image pickup device to pick up an endoscope image.

A memory 21a is provided in the control section 21. The memory 21a stores information about a power source to be used in the electric circuit 22. For example, the memory 21a stores information about a power source such as information about a voltage specification range of the electric circuit 22 such as the image pickup device. The control section 21 sends the information stored in the memory 21a to the endoscope processor 10 via a communication circuit 25.

The endoscope processor 10 includes a control section 11, a power source IC 12, a power setting section 13, a communication circuit 15, an endoscope connection section 17, a switch SW, and a discharge resistor R1. Note that in FIG. 1, illustration of configurations other than those related to power supply to the endoscope 20 is omitted.

The control section 11 controls the entirety of the endoscope processor 10. The control section 11 may be configured with an FPGA, or may be configured with a processor using an unshown CPU or the like, to be capable of controlling each section according to a program stored in the memory.

The communication circuit 15 receives information from the communication circuit 25 of the endoscope 20, and outputs the information to the control section 11. A memory 11a is provided in the control section 11. The control section 11 stores, in the memory 11a, the information received from the endoscope processor 10, i.e., information about the power source to be used in the electric circuit 22 of the endoscope 20. Based on the information stored in the memory 11a, the control section 11 generates information about an allowable range (outputtable range) of voltage that is suppliable to the electric circuit 22 such as the image pickup device, or about how the voltage is to be supplied to the electric circuit 22, for example, startup sequence information about the startup sequence and/or end sequence information about the end sequence, and stores the information in the memory 11a. At the times of startup and ending, the control section 11 is designed to set the power source voltage to be supplied to the endoscope 20, based on the startup sequence information and the end sequence information stored in the memory 11a.

The power setting section 13 is controlled by the control section 11, to perform setting for causing the power source IC 12 to generate a power source voltage to be supplied to the endoscope 20. For example, the power setting section 13 may be configured with a D/A converter or the like, to convert a control value from the control section 11 into an analog signal to be supplied to the power source IC 12.

The power setting section 13 causes a voltage according to the startup sequence to be output from the power source IC 12 at the startup of the endoscope apparatus, and causes a voltage according to the end sequence to be output from the power source IC 12 at ending of the endoscope apparatus. The power source IC 12, as a power source circuit, is controlled by the power setting section 13, to generate power having a voltage and a current that are specified by the control section 11. The output from the power source IC 12 is designed to be transmitted to the endoscope 20 via the switch SW.

The switch SW, as a power conduction circuit, is controlled by the control section 11, to be in a conduction state or a non-conduction state, to electrically connect or disconnect between the power source IC 12 and the endoscope 20. In other words, the switch SW connects or disconnects between the power source IC 12 and the scope power source 23 of the endoscope 20. When the switch SW is turned on, power from the power source IC 12 is supplied to the scope power source 23, and when the switch SW is turned off, power supply from the power source IC 12 to the scope power source 23 is cut off.

An output terminal of the power source IC 12 is grounded through the discharge resistor R1. It is designed such that, at the startup of the endoscope apparatus, a check, for example, abnormality detection, is performed on the power source IC 12, in order to determine whether power supply according to the startup sequence is possible. At the time when the power source IC 12 is checked, the switch SW is off. This makes it possible to inhibit a voltage for checking from being supplied to the scope side at the time when the power source IC 12 is checked. After the checking of the power source IC 12 ends, electric charge in the output terminal of the power source IC 12 is discharged by the discharge resistor R1.

### Endoscope Discharge Method in Comparative Example

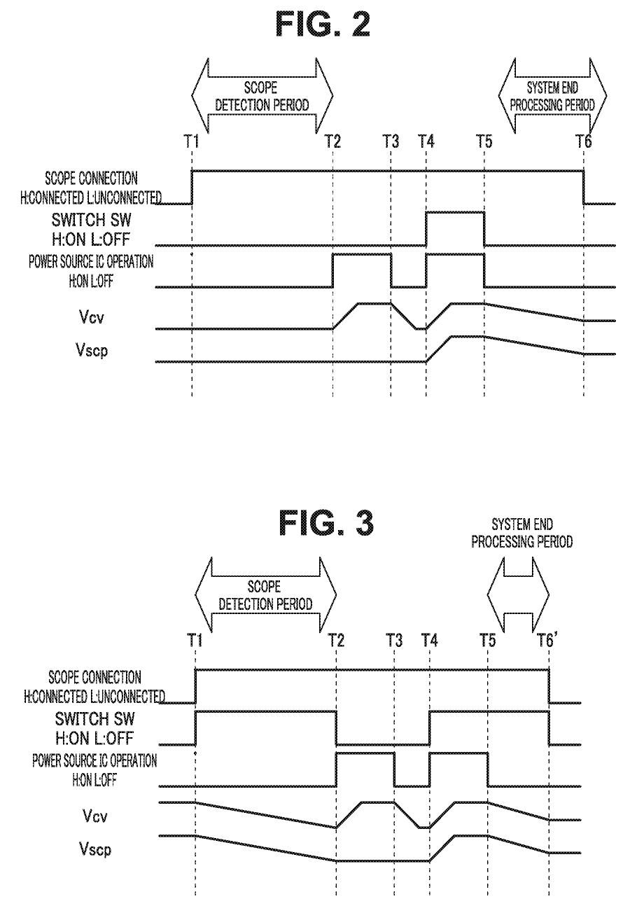

FIG. 2 is a timing chart showing an endoscope discharge method in a comparative example. FIG. 2 shows a scope connection state, on/off of the switch SW, on/off of the operation of the power source IC 12, a voltage Vcv at the output terminal of the power source IC 12, and a voltage Vscp at the scope power source 23. The example in FIG. 2 is one that assumes that the electric charge in the scope power source 23 is completely discharged at the startup of the endoscope apparatus. To ensure this assumption, the example in FIG. 2 is one in which the electric charge in the scope power source 23 is completely discharged at the ending of the endoscope apparatus.

Scope connection in FIG. 2 is indicated by a high level (hereafter referred to as "H") to indicate a connected state, and a low level (hereafter referred to as "L") to indicate an unconnected (disconnected) state. This state of scope connection is detected by the detection section of the control section 11, which detects that the endoscope 20 is connected to the endoscope processor 10. In the scope connection detection by the control section 11, determination of a type of the endoscope 20 connected, or the like, is also performed, and a predetermined period (hereinafter referred to as the "scope detection period") is necessary even after connection of the endoscope 20 is detected.

Assume that, at time T1 in FIG. 2, the endoscope 20 is connected. Then, within a predetermined scope detection period, the control section 11 of the endoscope processor 10 detects that the endoscope 20 is connected. Note that, at this time T1, as shown in FIG. 2, the switch SW is off and the power source IC 12 has not started operation (off). At time T1, the voltages Vcv and Vscp are 0 V.

When the scope detection period ends at time T2, the control section 11 starts (turns on) the operation of the power source IC 12, to perform operation check of the power source IC 12. This causes an output voltage Vcv of the power source IC 12 to increase and reach a specified voltage. When the operation check of the power source IC 12 ends at time T3, the control section 11 stops (turns off) the operation of the power source IC 12. This causes the voltage Vcv to gradually decrease to return to 0 V.

In a period from time T1 to time T4, where time T4 is after the voltage Vcv has returned to 0 V, the switch SW is off. This inhibits the voltage Vcv generated by the operation check of the power source IC 12 from being supplied to the endoscope 20, and power unrelated to the startup sequence from being supplied to the endoscope 20.

The electric charge in the output terminal of the power source IC 12 is discharged via the discharge resistor R1. As a result, the voltage Vcv returns to 0 V in a relatively short time after time T3. This enables the endoscope 20 to operate normally in a relatively short time.

When next time T4 is reached, the control section 11 turns on the power source IC 12 and also turns on the switch SW in order to cause the endoscope 20 to operate normally. This causes the voltage Vcv from the power source IC 12 to gradually rise. The voltage Vcv from the power source IC 12 is supplied to the scope power source 23 of the endoscope 20 via the switch SW. In this manner, the voltage Vscp of the scope power source 23 also gradually rises. When the output voltage Vcv of the power source IC 12 becomes the specified voltage and the voltage Vscp becomes a specified voltage, the endoscope 20 may operate normally.

The period of times T4 to T5 in FIG. 2 is a normal operation period of the endoscope 20, and during this period, an endoscopic examination is performed. When time T5, at which the examination ends, is reached, transition is made to the system end processing period. That is, at time T5, the control section 11 turns off the power source IC 12 and also turns off the switch SW. This causes both the voltages Vcv and Vscp to gradually decrease. The voltage Vscp of the scope power source 23 of the endoscope 20 decreases little by little due to natural discharge. Note that the voltage Vcv may be discharged in a shorter time than the voltage Vscp.

In the comparative example in FIG. 2, the voltage Vscp of the scope power source 23 is designed to decrease by natural discharge, and it takes a relatively long time for the voltage Vscp to become 0 V. That is, a relatively long time is necessary as the end processing period of the system. Hypothetically, if it is not possible to sufficiently ensure the end processing period, the electric charge in the scope power source 23 may remain without being completely discharged. Then, it may be necessary to discharge the scope power source 23 at the startup of the endoscope apparatus, resulting in a long startup time.

### Endoscope Discharge Method in Embodiment

Therefore, in the present embodiment, the control of the switch SW is modified to utilize an existing discharge resistor R1 for discharging the scope power source 23, thereby enabling the startup time and the end processing period to be shortened.

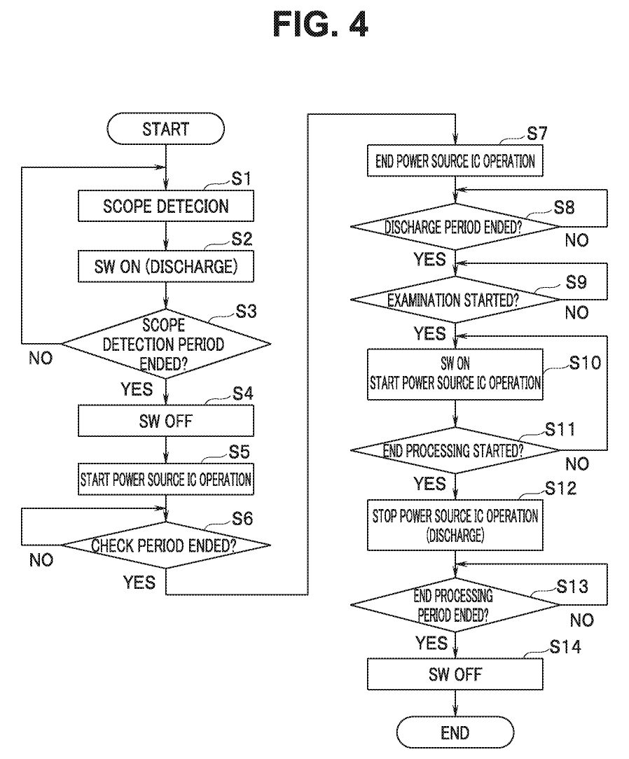

FIG. 3 is a timing chart showing the endoscope discharge method in the present embodiment. FIG. 3 shows the scope connection state, the on/off of the switch SW, the on/off of the operation of the power source IC 12, the voltage Vcv at the output terminal of the power source IC 12, and the voltage Vscp at the scope power source 23, using the same notation manner as in FIG. 2. The example in FIG. 3 is one for a case in which the electric charge in the scope power source 23 is not completely discharged at the startup of the endoscope apparatus. That is, FIG. 3 is an example where the electric charge in the scope power source 23 is not completely discharged, for example, without sufficiently ensuring the end processing period at the ending of the endoscope apparatus. FIG. 4 is a flowchart showing the endoscope discharge method in the present embodiment. It is noted that the control section 11 may be configured to execute a program 11b (see FIG. 1) stored in the memory 11a, thereby performing the process illustrated in FIG. 4.

When the power source voltage is supplied to the control section 11 of the endoscope processor 10, the control section 11, as an endoscope connection detection section, performs connection detection (scope detection) of whether the endoscope 20 is connected (S1 in FIG. 4). When detecting that the endoscope 20 is connected, the control section 11 turns on the switch SW (S2). Therefore, at time T1 in FIG. 3, as soon as the endoscope 20 is connected, the scope power source 23 is connected to a ground point via the switch SW and the discharge resistor R1, and the discharge of the electric charge in the scope power source 23 is started.

In the example in FIG. 3, at time T1, the electric charge at Vscp of the scope power source 23 is not completely discharged, and the voltages Vcv and Vscp are at predetermined voltage levels. However, in the present embodiment, during the scope detection period, the electric charge in the scope power source 23 is discharged via a discharge path by the switch SW and the discharge resistor R1, and the voltages Vcv and Vscp become 0 V by the end of the scope detection period, i.e., the start of driving the power source IC 12.

When end time T2 of the scope detection period is reached, the control section 11 determines the end of the scope detection period in S3. The control section 11 turns off the switch SW in next S4, and starts operation of the power source IC 12 (S5) to perform a check for abnormality detection, or the like. In this operation check, the output voltage Vcv of the power source IC 12 increases to reach a specified voltage. During this operation check, the switch SW is off, and the voltage Vcv generated by the operation check of the power source IC 12 is not supplied to the endoscope 20, and power unrelated to the startup sequence is not supplied to the endoscope 20.

When determining that the operation check of the power source IC 12 has ended at time T3 (S6), the control section 11 stops (turns off) the operation of the power source IC 12 (S7). The electric charge in the output terminal of the power source IC 12 is discharged via the discharge resistor R1. As a result, the voltage Vcv returns to 0 V in a relatively short time after time T3. This enables the endoscope 20 to operate normally in a relatively short time.

When determining in S8 that the discharge at the output terminal of the power source IC 12 is complete, the control section 11 determines in next S9 whether examination start is instructed. When the start of the endoscopic examination is instructed at time T4, the control section 11 turns on the power source IC 12 and also turns on the switch SW (S10), in order to cause the endoscope 20 to operate normally. This causes the voltage Vcv from the power source IC 12 to gradually rise. The voltage Vcv from the power source IC 12 is supplied from the switch SW to the scope power source 23 of the endoscope 20. In this manner, the voltage Vscp of the scope power source 23 also gradually rises. The output voltage Vcv of the power source IC 12 becomes the specified voltage and the voltage Vscp becomes the specified voltage, enabling the endoscope 20 to operate normally.

The period of times T4 to T5 in FIG. 3 is a normal operation period of the endoscope 20, and during this period, the endoscopic examination is performed. The control section 11 determines that the end of the examination is instructed (S11), and when time T5, at which the examination ends, is reached, transition is made to the system end processing period. That is, the control section 11 turns off the power source IC 12 at time T5 (S12).

In the present embodiment, the control section 11 keeps the switch SW on during the end processing period, i.e., even after the power source IC 12 stops driving. Therefore, during the end processing period, an input terminal of the scope power source 23 is connected to the ground point via the discharge path by the switch SW and the discharge resistor R1, and the electric charge in the scope power source 23 is discharged rapidly through the discharge path. In this manner, both the voltages Vcv and Vscp decrease relatively rapidly.

The control section 11 determines in S13 the end of the end processing period, and after the endoscopic examination, when the end processing period at time T6' is reached, for example, when the endoscope is removed or the power source supply is stopped, the control section 11 turns off the switch SW to end the processing. Note that the end processing period is preset depending on respective times required to completely discharge charge from the scope power sources 23 of various endoscopes 20 connectable to the endoscope processor 10, during the scope detection period and the end processing period.

Thus, in the present embodiment, the switch SW and the discharge resistor R1, which are existent, are utilized to turn on the switch SW and discharge the electric charge in the scope power source 23 via the discharge path by the discharge resistor R1, during the scope detection period and the end processing period, i.e., idle periods of the system operation. This enables the voltage Vscp to become 0 V in a shorter time than when the electric charge in the scope power source 23 is discharged by natural discharge. Therefore, a relatively short time may be set as the end processing period of the system, thereby inhibiting prolongation of the waiting time until removal of the endoscope. Hypothetically, even if the scope power source 23 cannot be completely discharged within the end processing period, the scope power source 23 may be discharged during the scope detection period at the startup of the endoscope apparatus, thereby inhibiting prolongation of the startup time and shortening the image output time of the endoscope.

### Second Embodiment

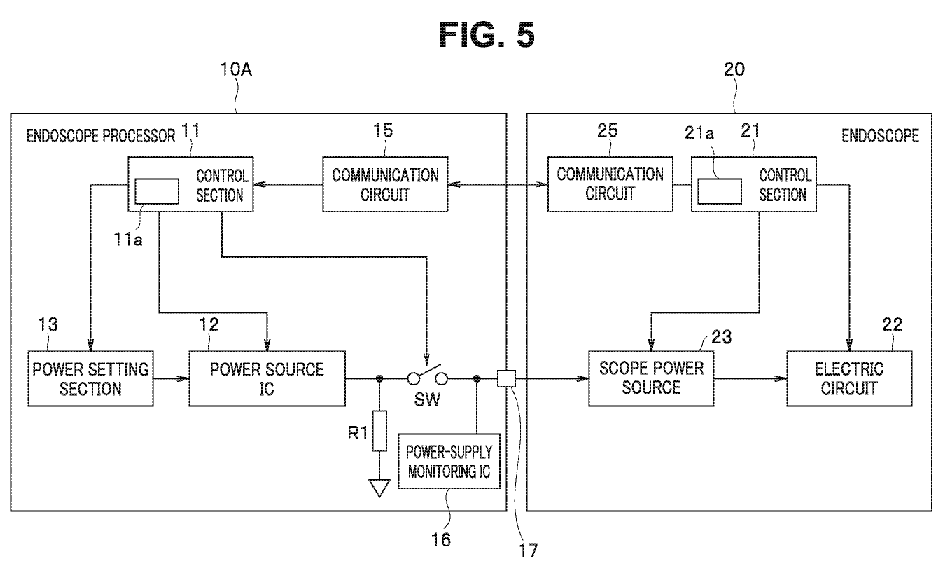

FIG. 5 is a configuration diagram showing an endoscope apparatus employing an endoscope discharge method according to the second embodiment of the present disclosure. In the first embodiment, it is designed such that the electric charge in the scope power source 23 of the endoscope 20 is discharged during the scope detection period and the end processing period that are preset. However, it is considered that, hypothetically when a single failure, such as poor connection of the discharge resistor R1, occurs, the discharge may not be completely performed. Therefore, in the present embodiment, a power-source monitoring IC 16 is employed to reliably enable the power source to be turned on according to the startup sequence.

An endoscope processor 10A of FIG. 5 differs from the endoscope processor 10 of FIG. 1 in that the power-source monitoring IC 16 is added. The power-source monitoring IC 16 is connected to a wiring between the switch SW and the scope power source 23, and is designed to monitor a voltage at the input terminal of the scope power source 23 and supply a monitoring result to the control section 11 (illustration omitted). Based on the monitoring result from the power-source monitoring IC 16, the control section 11 determines whether the voltage Vscp has become 0 V between times T2 and T4 in FIG. 3. The control section 11 is designed not to start the operation of the power source IC 12 until the voltage Vscp becomes 0 V.

That is, whereas in the first embodiment, time T4, which indicates endoscopy start time, is the time after a predefined period after the end of the operation check of the power source IC 12, in the present embodiment, the control section 11 sets, as the examination start time T4, the time after the voltage Vscp has become 0 V after the end of the operation check of the power source IC 12.

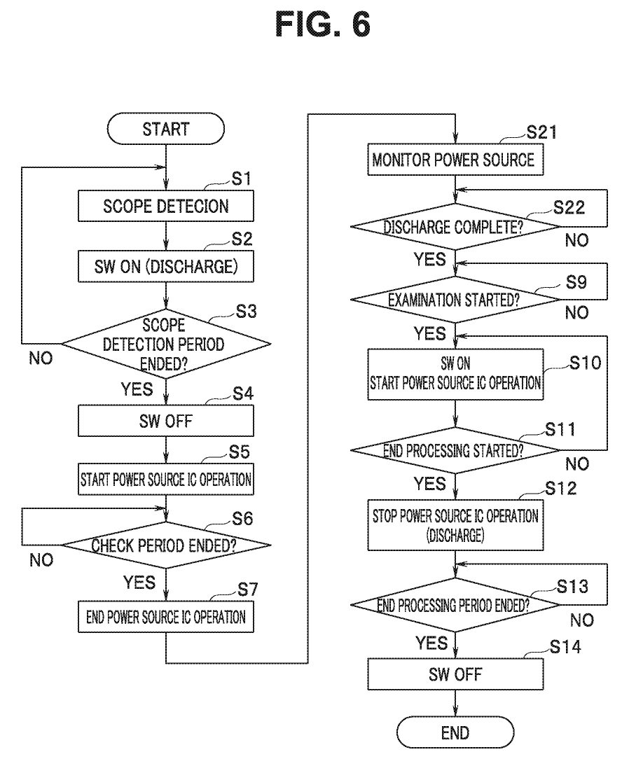

Next, an operation of an embodiment thus configured will be described with reference to FIG. 6. FIG. 6 is a flowchart for describing the second embodiment. In FIG. 6, steps same as those in FIG. 4 are marked with the same reference numerals, and descriptions thereof will be omitted.

The flow in FIG. 6 differs from the flow in FIG. 4 in that a power-source monitoring processing in step S21 is added, and a discharge completion determination in step S22 is employed instead of the discharge period end determination in step S8.

When stopping the operation of the power source IC 12 in S7, the control section 11 monitors a state of the power source at the input terminal of the scope power source 23 using the monitoring result from the power-source monitoring IC 16 (S21). The control section 11 determines, in S22, whether the discharge at the input terminal of the scope power source 23 is complete, based on the monitoring result from the power-source monitoring IC 16. The control section 11 does not proceed to next step S9 until the discharge at the input terminal of the scope power source 23 is complete. When determining that the discharge at the input terminal of the scope power source 23 is complete, the control section 11 determines, in S9, whether examination start is instructed. This ensures that, when the examination is started, a state is obtained in which the discharge at the input terminal of the scope power source 23 is complete, and the power source may be turned on according to the startup sequence.

Thus, the present embodiment yields the same effects as in the first embodiment, and has an advantage of reliably enabling the power source to be turned on according to the startup sequence even when a single failure or the like occurs.

The present disclosure is not limited to the above embodiments as they are, but may be embodied by transforming the constituent elements in the implementation stage within a scope not departing from the gist thereof. A plurality of constituent elements disclosed in each of the above embodiments may be combined as appropriate to form a variety of technical concepts. For example, some constituent elements of all the constituent elements shown in the embodiments may be deleted. Further, constituent elements across different embodiments may be combined as appropriate. Of the techniques described herein, many of the controls and functions, which are described mainly in the flowchart, may be set by a program, and the program may be read and executed by a computer to realize the controls and functions mentioned above.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another. Similarly, the term “non-transitory computer-readable (storage) medium” encompasses a single storage medium or a group of multiple storage media, which may be locally or remotely distributed and may collectively store and provide access to instructions, data, or other information in a coordinated or distributed manner.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:

Clause 1: An endoscope processor comprising: a power-source supply circuit configured to supply power to an endoscope; an endoscope connection section to which the endoscope is connectable; a discharge resistor placed between the power-source supply circuit and the endoscope connection section; a power-source conduction circuit placed between the discharge resistor and the endoscope connection section; and a processor configured to: detect that the endoscope is connected to the endoscope connection section; control start and stop of power supply by the power-source supply circuit; and control conduction and non-conduction by the power-source conduction circuit, wherein the processor is configured to: when the endoscope is connected to the endoscope connection section, control the power-source conduction circuit to establish conduction between the endoscope and the discharge resistor; upon start of power supply to the endoscope, control the power-source conduction circuit to cause non-conduction between the endoscope and the discharge resistor; and upon resumption of power supply to the endoscope after a stop, control the power-source conduction circuit to establish conduction between the endoscope and the discharge resistor, and wherein, when the endoscope is in conduction with the discharge resistor, the processor is configured to cause electric charge accumulated in the endoscope to be discharged from the discharge resistor of the endoscope processor.

Clause 2: The endoscope processor according to clause 1, wherein the processor is configured to, after endoscopy ends, stop driving the power-source supply circuit, and then bring the power-source conduction circuit into a conduction state, thereby establishing conduction between the endoscope and the discharge resistor, to discharge electric charge accumulated in the endoscope from the discharge resistor of the endoscope processor.

Clause 3: The endoscope processor according to clause 1, wherein the processor is configured to, after the endoscope is connected to the endoscope connection section to bring the power-source conduction circuit into a conduction state, bring the power-source conduction circuit into a non-conduction state, while driving the power-source supply circuit to perform abnormality detection on the power-source supply circuit.

Clause 4: The endoscope processor according to clause 1, wherein the processor is configured to bring the power-source conduction circuit into a conduction state, upon detection that the endoscope changes from a disconnected state to a connected state.

Clause 5: An endoscope discharge method for an endoscope processor including a processor, wherein the processor is configured to: when an endoscope is connected to an endoscope connection section to which the endoscope is connectable, control a power-source conduction circuit to establish conduction between the endoscope and a discharge resistor, the power-source conduction circuit being placed between the discharge resistor and the endoscope connection section, the discharge resistor being placed between a power-source supply circuit and the endoscope connection section, the power-source supply circuit being configured to supply power to the endoscope; upon start of power supply to the endoscope, control the power-source conduction circuit to cause non-conduction between the endoscope and the discharge resistor; and upon resumption of power supply to the endoscope after a stop, control the power-source conduction circuit to establish conduction between the endoscope and the discharge resistor, and wherein, when the endoscope is in conduction with the discharge resistor, the processor causes electric charge accumulated in the endoscope to be discharged from the discharge resistor of the endoscope processor.

Clause 6: The endoscope discharge method according to clause 5, wherein the processor is configured to, after endoscopy ends, from when the power-source supply circuit stops driving until the endoscope is removed, bring the power-source conduction circuit into a conduction state, establish conduction between the endoscope and the discharge resistor, thereby discharging electric charge accumulated in the endoscope from the discharge resistor of the endoscope processor.

Clause 7: The endoscope discharge method according to clause 5, wherein the processor is configured to, after the endoscope is connected to the endoscope connection section to bring the power-source conduction circuit into a conduction state, bring the power-source conduction circuit into a non-conduction state while driving the power-source supply circuit to perform abnormality detection on the power-source supply circuit.

Clause 8: A computer-readable medium storing an endoscope discharge program that causes a computer to execute steps comprising: when an endoscope is connected to an endoscope connection section to which the endoscope is connectable, controlling a power-source conduction circuit to establish conduction between the endoscope and a discharge resistor, the power-source conduction circuit being placed between the discharge resistor and the endoscope connection section, the discharge resistor being placed between a power-source supply circuit and the endoscope connection section, the power-source supply circuit being configured to supply power to the endoscope; upon start of power supply to the endoscope, controlling the power-source conduction circuit to cause non-conduction between the endoscope and the discharge resistor; and upon resumption of power supply to the endoscope after a stop, controlling the power-source conduction circuit to establish conduction between the endoscope and the discharge resistor, wherein when the endoscope is in conduction with the discharge resistor, electric charge accumulated in the endoscope is discharged from the discharge resistor of the endoscope processor.

What is claimed is:

1. An endoscope processor comprising:
a power source circuit configured to supply power to an endoscope;
an endoscope connector;
a discharge resistor between the power source circuit and the endoscope connector;
a power conduction circuit between the discharge resistor and the endoscope connector; and
a processor configured to:
in response to detecting connection of the endoscope to the endoscope connector, control the power conduction circuit to establish conduction between the endoscope and the discharge resistor;
in response to starting power supply to the endoscope by the power source circuit, control the power conduction circuit to interrupt the conduction; and
in response to resuming power supply to the endoscope after a stop, control the power conduction circuit to reestablish the conduction,
wherein, when the conduction is established, charge accumulated in the endoscope is discharged through the discharge resistor.

2. The endoscope processor according to claim 1, wherein the processor is further configured to, after an end of an endoscopic examination, stop driving the power source circuit, and set the power conduction circuit to a conductive state where the conduction is established between the endoscope and the discharge resistor, thereby discharging charge accumulated in the endoscope through the discharge resistor.

3. The endoscope processor according to claim 1, wherein the processor is further configured to, after detecting connection of the endoscope to the endoscope connector and setting the power conduction circuit to a conductive state, set the power conduction circuit to a non-conductive state, while driving the power source circuit to perform abnormality detection on the power source circuit.

4. The endoscope processor according to claim 1, wherein the processor is further configured to set the power conduction circuit to a conductive state in response to detecting a change from a first state where the endoscope is not connected to the endoscope connector to a second state where the endoscope is connected to the endoscope connector.

5. The endoscope processor according to claim 1, wherein the processor is further configured to set the power conduction circuit to a conductive state during a first period after the endoscope is connected to the endoscope connector, thereby connecting a scope power source of the endoscope to ground via the power conduction circuit and the discharge resistor.

6. The endoscope processor according to claim 5, wherein the processor is further configured to, during a second period following the first period, drive the power source circuit and set the power conduction circuit to a non-conductive state, thereby performing abnormality detection on the power source circuit.

7. The endoscope processor according to claim 6, wherein the processor is further configured to:
during a third period following the second period, stop driving the power source circuit and maintain the non-conductive state of the power conduction circuit, thereby discharging charge from an output terminal of the power source circuit through the discharge resistor;
during a fourth period following the third period, drive the power source circuit and set the power conduction circuit to the conductive state, thereby enabling an endoscopic examination with the endoscope; and
during a fifth period following the fourth period, stop driving the power source circuit and maintain the conductive state of the power conduction circuit, thereby discharging charge accumulated in the endoscope through the discharge resistor.

8. The endoscope processor according to claim 7, wherein the first period and the fifth period are set in accordance with respective times required to discharge charge from scope power supplies of various types of endoscopes connectable to the endoscope connector.

9. The endoscope processor according to claim 1, further comprising a communication circuit,
wherein the processor is further configured to:
receive power source information from the endoscope via the communication circuit, the power source information being information about a power source used in an electric circuit of the endoscope; and
set startup sequence information and/or end sequence information based on the received power source information.

10. The endoscope processor according to claim 1, further comprising a power-source monitoring IC disposed between the power conduction circuit and a scope power source of the endoscope and configured to monitor a voltage at an input terminal of the scope power source,
wherein the processor is further configured to:
receive a monitoring result from the power-source monitoring IC;
determine whether discharge at the input terminal of the scope power source is complete based on the received monitoring result; and
inhibit driving the power source circuit to start an endoscopic examination until discharge at the input terminal of the scope power source is complete.

11. A method implementable on a processor of an endoscope processor comprising a power source circuit, an endoscope connector, a discharge resistor, and a power conduction circuit, the method comprising:
in response to detecting connection of an endoscope to the endoscope connector, controlling the power conduction circuit to establish conduction between the endoscope and a discharge resistor, the power conduction circuit being disposed between the discharge resistor and the endoscope connector, the discharge resistor being disposed between the power source circuit and the endoscope connector, the power source circuit being configured to supply power to the endoscope;
in response to starting power supply to the endoscope by the power source circuit, controlling the power conduction circuit to interrupt the conduction; and
in response to resuming power supply to the endoscope after a stop, controlling the power conduction circuit to reestablish the conduction,
wherein, when the conduction is established, charge accumulated in the endoscope is discharged through the discharge resistor.

12. The method according to claim 11, further comprising:
after an end of an endoscopic examination, from when the processor stops driving the power source circuit until the endoscope is removed, setting the power conduction circuit to a conductive state where the conduction is established between the endoscope and the discharge resistor, thereby discharging charge accumulated in the endoscope through the discharge resistor.

13. The method according to claim 11, further comprising:
after detecting connection of the endoscope to the endoscope connector and setting the power conduction circuit to a conductive state, setting the power conduction circuit to a non-conductive state, while driving the power source circuit to perform abnormality detection on the power source circuit.

14. The method according to claim 11, further comprising:
setting the power conduction circuit to a conductive state during a first period after the endoscope is connected to the endoscope connector, thereby connecting a scope power source of the endoscope to ground via the power conduction circuit and the discharge resistor.

15. The method according to claim 14, further comprising:
during a second period following the first period, driving the power source circuit and setting the power conduction circuit to a non-conductive state, thereby performing abnormality detection on the power source circuit.

16. The method according to claim 15, further comprising:
during a third period following the second period, stopping driving the power source circuit and maintaining the non-conductive state of the power conduction circuit, thereby discharging charge from an output terminal of the power source circuit through the discharge resistor;
during a fourth period following the third period, driving the power source circuit and setting the power conduction circuit to the conductive state, thereby enabling an endoscopic examination with the endoscope; and
during a fifth period following the fourth period, stopping driving the power source circuit and maintaining the conductive state of the power conduction circuit, thereby discharging charge accumulated in the endoscope through the discharge resistor.

17. The method according to claim 16, wherein the first period and the fifth period are set in accordance with respective times required to discharge charge from scope power supplies of various types of endoscopes connectable to the endoscope connector.

18. The method according to claim 11,
wherein the endoscope processor further comprises a communication circuit, and
wherein the method further comprises:
receiving power source information from the endoscope via the communication circuit, the power source information being information about a power source used in an electric circuit of the endoscope; and
setting startup sequence information and/or end sequence information based on the received power source information.

19. The method according to claim 11,
wherein the endoscope processor further comprises a power-source monitoring IC disposed between the power conduction circuit and a scope power source of the endoscope and configured to monitor a voltage at an input terminal of the scope power source,
wherein the method further comprises:
receiving a monitoring result from the power-source monitoring IC;
determining whether discharge at the input terminal of the scope power source is complete, based on the received monitoring result; and
inhibiting driving the power source circuit to start an endoscopic examination until discharge at the input terminal of the scope power source is complete.

20. A non-transitory computer-readable medium storing computer-readable instructions that are executable by a processor of an endoscope processor comprising a power source circuit, an endoscope connector, a discharge resistor, and a power conduction circuit, the computer-readable instructions being configured to, when executed by the processor, cause the endoscope processor to execute the method according to claim 11.
